(19) United States
(12) Patent Application Publication
(10) Pub. No.: US 2017/0304081 A1
(43) Pub. Date: Oct. 26, 2017

(54) PROSTHESIS

(71) Applicant: OTTO BOCK HEALTHCARE GMBH, Duderstadt (DE)

(72) Inventors: Martin PUSCH, Duderstadt (DE); Christian WILL, Gottingen (DE); Herman BOITEN, Gottingen (DE)

(73) Assignee: OTTO BOCK HEALTHCARE GMBH, Duderstadt (DE)

(21) Appl. No.: 15/520,372

(22) PCT Filed: Oct. 14, 2015
(86) PCT No.: PCT/DE2015/100426

§ 371 Date: Apr. 19, 2017

(30) Foreign Application Priority Data
Oct. 27, 2014 (DE) 10 2014 015 756.3

(51) Int. Cl. A61F 2/60; A61F 2/64; A61F 2/50; A61F 2/76; A61F 2/30

(52) U.S. Cl. CPC A61F 2/60; A61F 2/64; A61F 2002/30546; A61F 2002/7625; A61F 2002/5073

(57) ABSTRACT

A prosthesis with a support part on which a pivot joint is secured or formed. A distal prosthesis component is secured to the support part via the pivot joint, and the pivot joint allows a flexion and extension of the distal prosthesis component about a pivot axis relative to the receiving element or to the support part. A damping device, spring device, and/or actuator is arranged between the distal prosthesis component and the support part, and the damping device, spring device, and/or actuator is mounted on the distal prosthesis component using a first bearing point and is mounted on a lever or an arm using a second bearing point. The lever is mounted on the distal prosthesis component in a pivotal manner about a pivot axis. A distal portion of the arm is mounted on the lever, and a proximal portion is mounted on the support part.

The invention relates to a prosthesis comprising a support part on which a swivel joint is formed or fastened. The prosthesis can be utilized as a replacement for missing upper and lower extremities, for example comprising a prosthetic foot, a prosthetic ankle joint and a below knee shaft, as an AKP (above knee prosthesis) comprising a femoral shaft, a prosthetic knee joint and a distal prosthesis component in the form of a below knee tube or a below knee part comprising connection elements for a prosthetic foot, or as a prosthesis for an upper extremity, for example comprising an elbow joint for hingedly connecting an upper arm socket to a forearm part which comprises receiving devices for a prosthetic hand. The prosthesis is not limited to the areas of application mentioned, although said prosthesis is particularly well suited therefor. The prosthesis is suitable, in particular, for the restoration of long stumps and exarticulation stumps, in which the swivel axis is located very close to the stump end or proximal to the stump end.

US 2012/0150318 A1 relates to a prosthesis for lower extremities, comprising a receiving element for receiving a femoral shaft, a support part comprising a swivel joint and a distal prosthesis component which is hingedly fastened to the support part via the swivel joint. The support part and the distal prosthesis component form the upper part and the lower part of a prosthetic knee joint. A connecting rod is fastened, in a posterior arrangement, to the support part via a radial arm, and the distal end of the connecting rod is coupled to a double-armed swiveling lever. The swiveling lever is swiveled, during flexion and extension of the prosthetic knee joint, about a swivel axis which is fixed on the below knee part. A damping device is situated on the side of the lever facing away from the swivel axis and is mounted, at its upper end, in the bearing point of the swivel joint or at the bearing points of the swivel joint. This prosthesis is used, in particular, as a ski prosthesis. The mounting of the damper in the axis of a joint results in a direct introduction of force into the upper part of the prosthetic joint.

The problem addressed by the present invention is that of providing a prosthesis which is suitable, in particular, for great stump lengths, and therefore exarticulation patients can also receive improved prosthetic restoration.

According to the invention, this problem is solved by a prosthesis having the features of the main claim. Advantageous embodiments and refinements of the invention are disclosed in the dependent claims, the description, and the figures.

In the prosthesis comprising a support part which can be used for fastening a receiving element, a swivel joint is fastened or formed on the support part, via which a distal prosthesis component is fastened to the support part. The swivel joint allows for a flexion and/or extension of the distal prosthesis component about an axis of the joint relative to the receiving element or the support part. A damping device for damping the flexion and/or the extension of the distal prosthesis component is situated between the distal prosthesis component and the support part or the receiving element. Alternatively or in addition to the damping device, a spring device or an actuator device, for example, a motor, a linear drive, a magnetic drive, or the like, can be situated between the distal prosthesis component and the support part in order to store or convert forces, or to introduce mechanical energy into the joint. The damping device, spring device and/or actuator device, or a combination thereof, are mounted on the distal prosthesis component by mean of a first bearing point and are mounted on a lever or a connecting rod by means of a second bearing point. In this case, the lever is mounted on the distal prosthesis component so as to swivel about a swivel axis. The connecting rod is mounted on the lever via a distal portion and is mounted on the support part or on the receiving element via a proximal portion.

Due to the articulated coupling of the damping device, spring device and/or actuator device via a lever joint, it is possible to utilize relatively small damping devices, spring device and/or actuator device, and to make full use of mechanical advantage, and therefore the relatively great swivel travel of the support part or the receiving element relative to the distal prosthesis component is reduced. If great displacement travel is required, for example, for spring-type accumulators, this can also be set via a corresponding mechanical advantage. In addition, it is possible to freely select a bearing point by way of the coupling of the damping device, spring device and/or actuator device in the distal prosthesis component, and therefore standard components such as dampers, springs, or motors, etc., can be utilized, which can be easily adapted to the particular individually adaptable prosthesis simply by adjusting the lever length, lever lengths, or connecting-rod lengths, and by way of the arrangement of the particular swivel axes. The lever arrangement makes it possible to position the damping device, spring device and/or actuator device within the distal prosthesis element in a manner which is geometrically favorable, in particular being optimized with respect to installation space.

Since the prosthesis according to the prior art allowed for a mounting of the damping device in the axis of the joint, it is necessary that the axis of the joint extend distally beyond the stump end or the distal end of the receiving element, or be situated at the distal end of the support part which, in turn, extends beyond the stump end. As a result, the axis of the joint according to the prior art must be situated distally far with respect to the natural axis of the joint if it was possible to achieve a relatively long stump, for example, in the case of joint exarticulations, in particular in the case of knee exarticulations. If the stump is formed with a bony structure having a condyle, this can be advantageous in terms of the loadability of the stump end. In the case of a restoration using a prosthesis according to the prior art, this would mean a change in the length of the restored leg in the upper leg region, which would be clearly noticeable in the sitting position, in particular. The solution according to the invention makes it possible to virtually freely select the axis of the joint, in particular to situate the axis of the joint in such a way that the stump length is minimized. As a result, it becomes possible to configure the restored limb using a length of the components that corresponds to the unrestored limb. In the case of a knee exarticulation prosthesis, this has the advantage that the length of the upper leg does not extend beyond that of an unrestored leg, which can pose a problem in public transportation. The same applies for aircraft, automobiles, and restaurant visits or the like, in which hindrances occur or unwanted collisions take place due to limb portions, for example, upper legs, that are longer as compared to the natural limb.

By way of the limb components, such as the upper leg and the lower leg, having the same length on the side fitted with a prosthesis and the side not fitted with a prosthesis, which can be achieved by means of the prosthesis according to the invention, it is possible to ride conventional bicycles without the need to adjust the crank lengths. Kneeling can take place uniformly, which, in the case of non-uniform upper legs, can only be accomplished at a slant and is usually associated with hip pain. In the case of prostheses for the upper extremities, the conspicuousness of the different upper-arm lengths is reduced; in the case of use with prosthetic feet, the prosthesis allows for a damped prosthetic foot even in the case of long below knee stump lengths which could not be fitted otherwise with a damped prosthetic foot.

The receiving element can be designed for receiving a stump and can be fastened to the support, wherein the receiving element can be designed as a conventional stump socket, as a liner-socket system and/or as a distal cup which can be fixed on the stump via a linkage or a stump receptacle. The receiving element can be fastened to the support part via a locking system, for example, via a pin system or a shuttle lock. The receiving element can be designed as a liner and can be fastened directly to the support part. The support part itself can be designed as a stump receptacle and can receive the stump directly or by means of a liner. The liner can be fastened to the support part via negative pressure and/or interlocking elements. In principle, it is also possible that an osseointegratable component is formed on or fastened to the support part and can be anchored in the bone.

The lever can be designed as a double-armed lever, wherein the swivel axis is positioned between the damping device and the connecting rod. As a result, it is possible to adjust the leverage ratio and, therefore, the displacement ratio between the damping device and the connecting rod in a simple and varied manner, which is reflected in the displacement and, therefore, also in the maximum displacement length of the damping device, spring device and/or actuator device. The greater the leverage ratio is, the lesser the displacement travel of the damping device, spring device and/or actuator device is. The damping device is advantageously designed as a hydraulic damper or a pneumatic damper or a combination of hydraulic damper and pneumatic damper.

As an alternative to a double-armed embodiment as a lever, similar to a rocker, this can also be designed as a single-armed lever, wherein the connecting rod and the damping device, spring device and/or actuator device are mounted on the lever at a distance from the swivel axis, and both the connecting rod and the damping device, spring device and/or actuator device are mounted on the one lever, which allows for a stable correlation and requires relatively little installation space.

The connecting rod and/or the damping device, spring device and/or actuator device can be displaceably mounted on the lever, and therefore an adjustment of the leverage ratios between the connecting rod and the damping device, spring device and/or actuator device can take place in order to accommodate needs or desires of the patient. For this purpose, receptacles for receiving the damping device, spring device and/or actuator device or the connecting rod can be situated and formed on the lever at discrete intervals or via slots, and therefore an adjustment of the position of the connecting rod and/or of the damping device, spring device and/or actuator device on the lever can take place and, once the adjustment has taken place, this geometric correlation can be fixed. The mobility of the connecting rod and the damping device, spring device and/or actuator device on the lever is also given due to the mounting situation.

Both the connecting rod and the damping device, spring device and/or actuator device, or the lever can be designed to be variable in length, for example, via threaded sleeves or telescopic receptacles which can be re-fixed in the particular desired position, and therefore a multiplicity of settings with respect to the length and, therefore, the leverage ratios between the damping device, spring device and/or actuator device, the connecting rod and the lever are possible and provided.

The distal prosthesis component can form a hollow space in which the lever and the damping device, spring device and/or actuator device are situated. The mechanical components of the prosthesis can be accommodated via this hollow space, for example, in the form of the missing distal limb, for example, a forearm or a lower leg. The distal prosthesis component can have the shape of the particular limb it is intended to replace. Due to the embodiment of the hollow space in the distal prosthesis component, an approximately natural impression, on the one hand, and a protected space for the movable mechanical components, on the other hand, are created. The distal prosthesis component therefore also offers mechanical protection of the lever arrangement and of the damping device, spring device and/or actuator device and further components such as sensors or a control system.

According to one advantageous refinement of the invention, the connecting rod at least partially covers or blocks the access to the hollow space. The hollow space comprises a cutout, in particular in the region in which the distal prosthesis component approaches the support part or the receiving element, through which the connecting rod can enter the hollow space of the distal prosthesis component and interact with the lever. Due to the rotary movement about the axis of the joint and due to the offset of the upper bearing point of the connecting rod with respect to the axis of the joint during the swinging motion of the distal prosthesis component, the lever executes a displacement on a circular path, which results not only in a longitudinal displacement of the connecting rod, but also a rotation about the bearing point on the lever. This means that the lever also executes a swinging motion relative to the distal prosthesis component, which makes a relatively large through-hole in the distal prosthesis component necessary. Due to an, e.g., planar embodiment of the connecting rod or the arrangement of a planar covering part on the, e.g., posterior part of the connecting rod, it is possible to complete the distal prosthesis component, with respect to the circumference, by way of the connecting rod and to at least partially cover the access to the hollow space, and therefore the connecting rod performs not only a force-transmission function but also a protective function. In the embodiment of the distal prosthesis component as a below knee tube, the connecting rod can be designed in the form of a calf or can supplement the distal prosthesis component in order to form a calf.

The axis of the joint, which is formed between the support part or the receiving element and the distal prosthesis component, advantageously extends through the support part or the receiving element, and therefore it is possible that the axis of the joint is positioned or can be positioned in the region of a natural axis of the joint of a limb. In this case, the axis of the joint can be positioned proximally to a distal end of the support part of the receiving element, which has advantages in terms of the particular arrangement of the axis of the joint in the case of long stumps.

The distal prosthesis component can be mounted directly on the support part or on the receiving part at at least one bearing point; the mounting advantageously takes place at two bearing points which lie on the axis of the joint. The at least one bearing point is located medially or laterally on the support part of the receiving element; when two bearing points are utilized, these bearing points are located medially and laterally on the support part and the receiving element.

The support part can be designed as a cap which can be connected to the receiving element in the shape of a socket or multiple rails. The stump can rest directly on or against, in the support part; it is also possible that the receiving element in the shape of a solid external shaft forms the upper part of the prosthetic joint and the distal prosthesis component is swivel-mounted directly on the receiving element.

The connecting rod can be displaceably mounted on the support part or on the receiving element, whereby stronger influences on the course of the damping curve, in particular the reversal point of the damping device, can be achieved.

Exemplary embodiments of the invention are explained in greater detail in the following on the basis of the attached figures. Wherein:

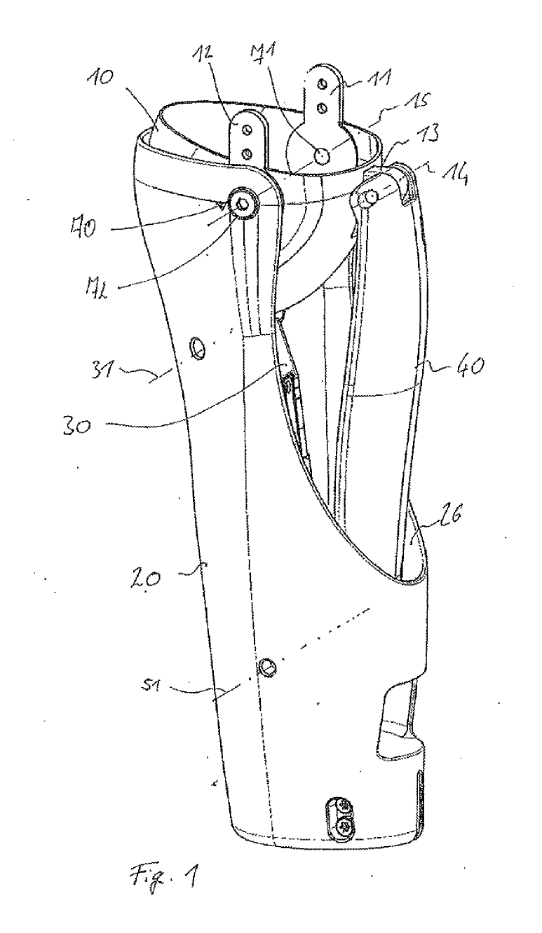

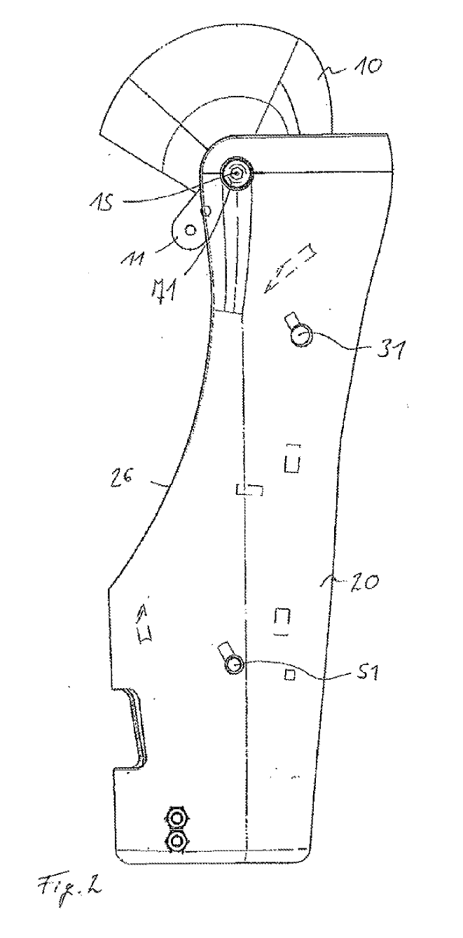

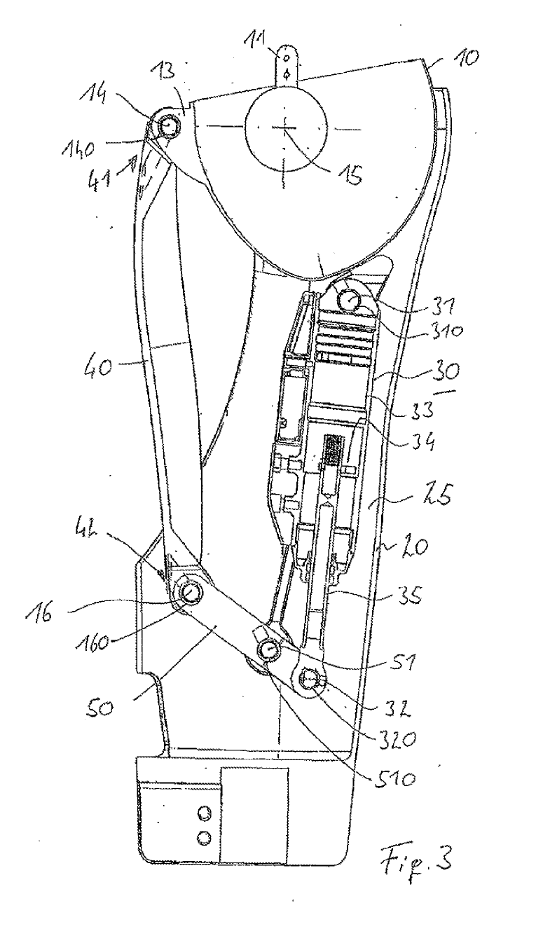

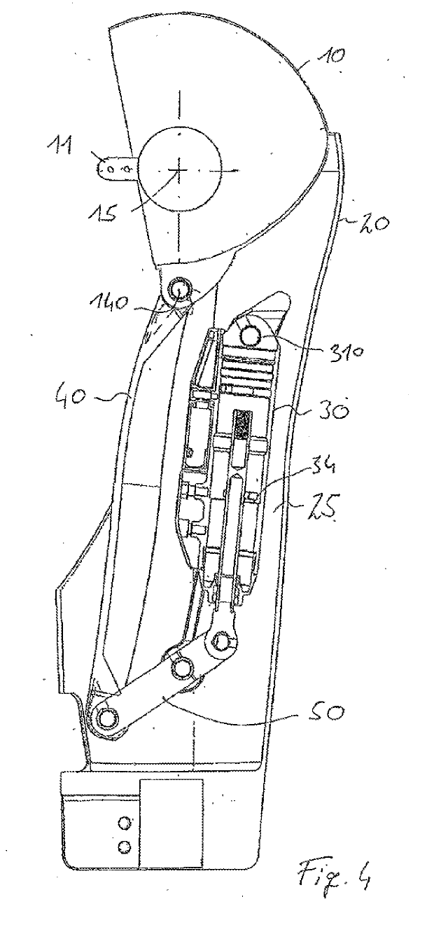

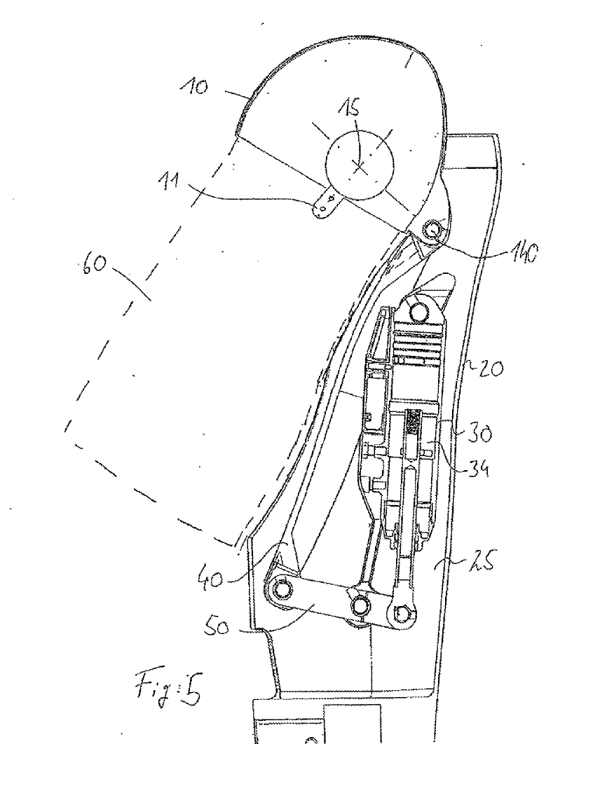

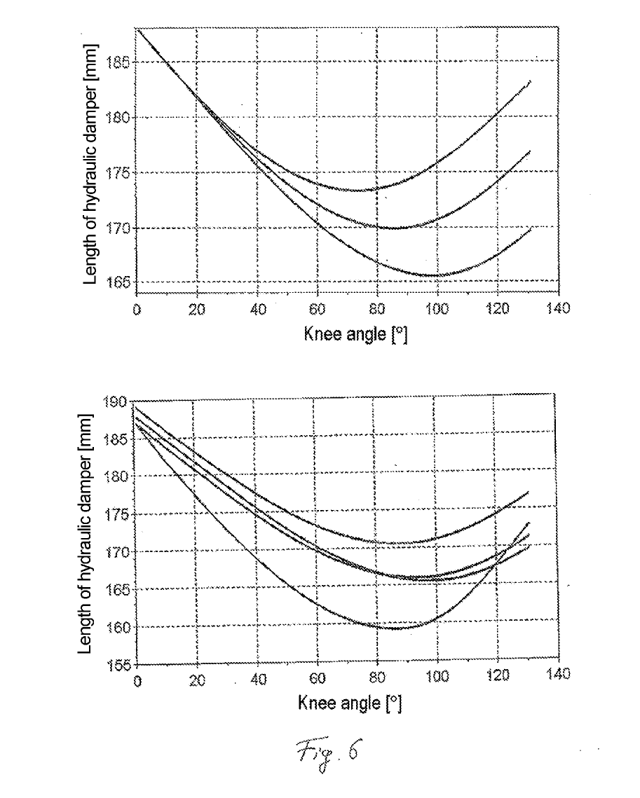

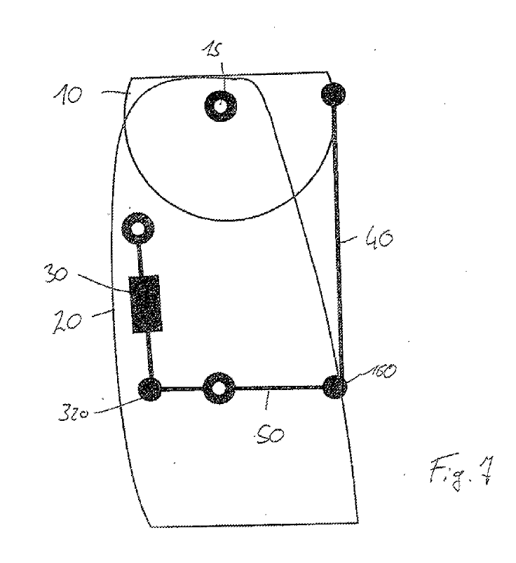

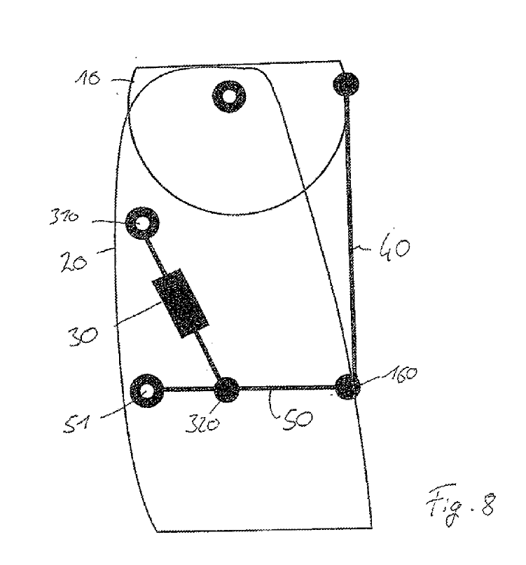

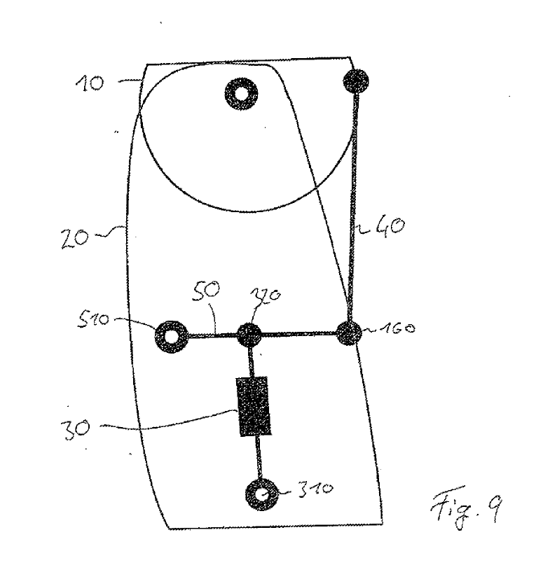

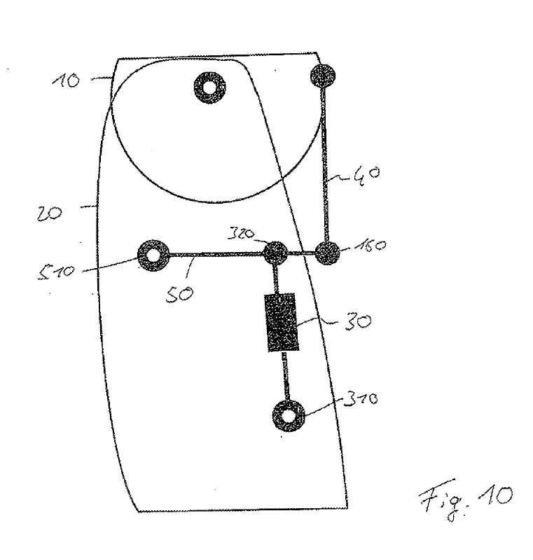

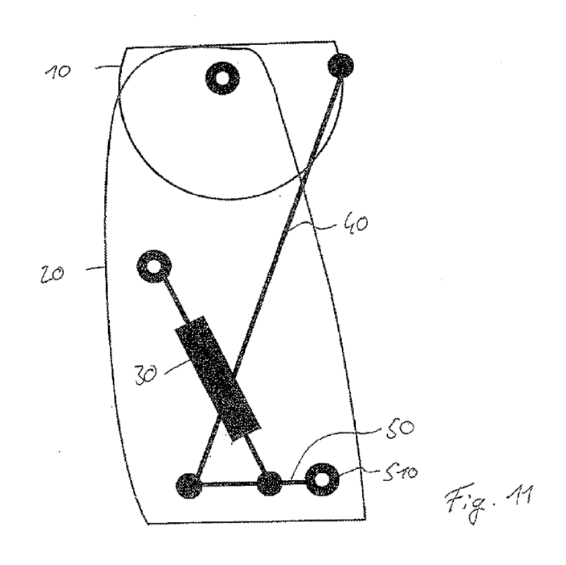

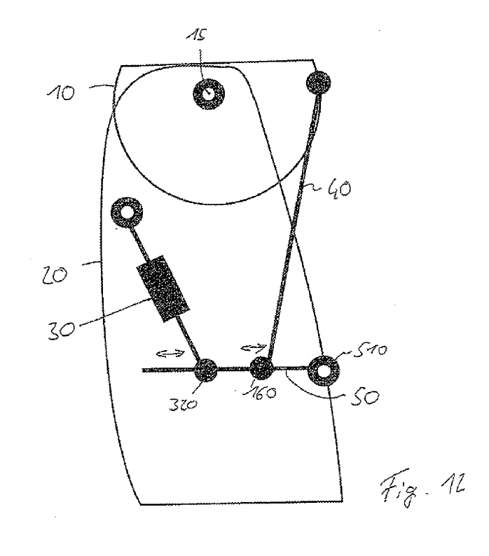

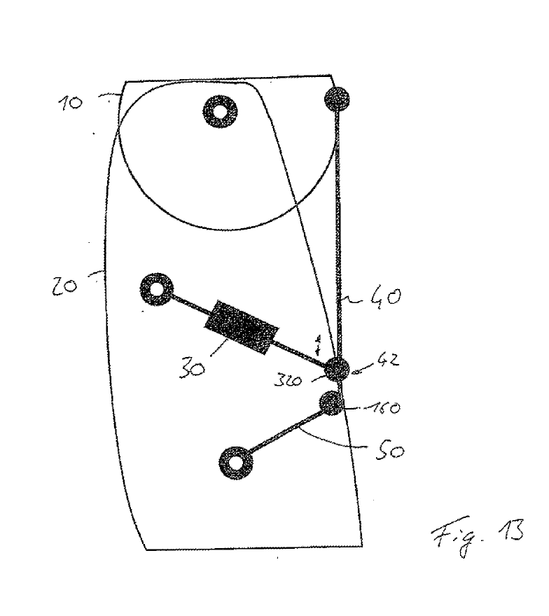

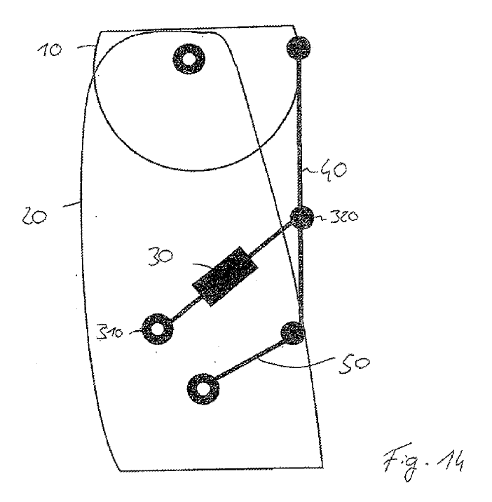

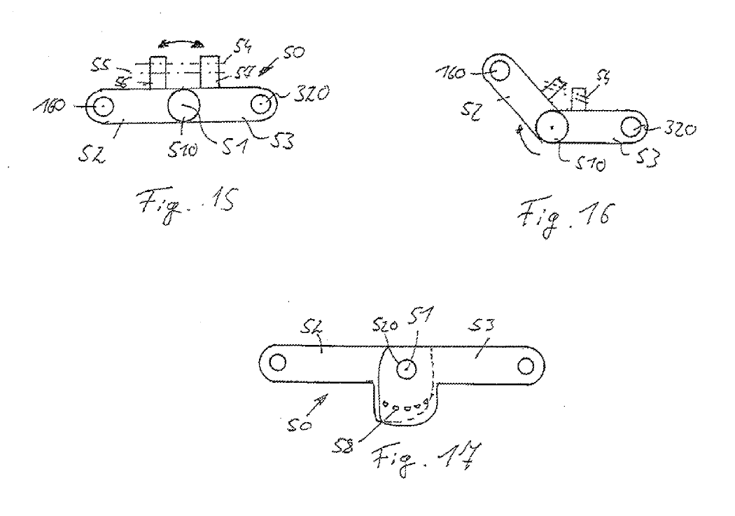

FIG. 1 shows a perspective view of a prosthesis;

FIG. 2 shows a side view of a prosthesis without a damper depicted;

FIG. 3 shows a sectional view through a prosthesis in the extension position;

FIG. 4 shows a prosthesis according to FIG. 3, in the 90° position;

FIG. 5 shows a depiction according to FIG. 5, in the maximally flexed position;

FIG. 6 shows damping curves for the knee angle as a function of the length of the hydraulic damper;

FIGS. 7 to 14 show schematic depictions of variants of the invention;

FIG. 15 shows a double-armed lever in a home position;

FIG. 16 shows the lever according to FIG. 15, in a displaced position; and

FIG. 17 shows one variant of a double-armed lever.

FIG. 1 shows a perspective depiction, obliquely from the rear, of a prosthesis in the form of a below knee prosthesis comprising a support part 10 on which a receiving element for receiving a stump is formed or can be fastened. The receiving element can be designed, for example, a prosthesis socket having a closed circumference, longitudinal braces which encircle an upper leg stump and are fixed by means of circular pulling means, or another receiving device. In principle, it is also possible that the support part 10 is designed for receiving the stump or the stump with a liner. In order to fasten a separate receiving element, medial and lateral fastening tabs 11, 12 are provided, which are fastened to the support part 10 and via which the receiving element or the receiving elements can be fastened to the support part 10. The fastening can be achieved via screws, bolts, or other interlocking elements; it is also possible that the receiving element is cemented into or onto the support part 10 which has a cup-like shape, or is fastened onto or in said support part in an interlocked or bonded manner in another way.

The support part 10 is fastened to a distal prosthesis component 20, i.e. a below knee part in the exemplary embodiment shown, so as to swivel about an axis of a joint 15. The axis of the joint 15 extends from medial to lateral and preferably through the tabs 11, 12. In order to provide for a swiveling fastening of the support part 10 to the distal prosthesis component 20, bearing points 72, 71 are formed medially and laterally on the distal prosthesis element 20 designed as a hollow body, and therefore a swivel joint 70 is formed between the support part 10 and the distal prosthesis component 20.

A damping device 30 is situated within the distal prosthesis component 20; in addition or as an alterative to the damping device 30, it is possible to provide a spring device, an actuator device, or a combination of at least two of these devices. The damping device 30 shown is designed as a hydraulic damper and comprises an upper bearing point which is proximal, i.e., is fastened in the proximity of the swivel joint 30, and which is described in greater detail further below. The upper or proximal bearing point forms a bearing axis 31 which is oriented essentially in parallel to the axis of the joint 15. If the damping device is mentioned in the following, the comments also apply for spring devices and/or actuator devices or combinations of two or all devices.

A connecting rod receptacle 13 is formed on the support part 10 on a rear posterior side and is used for accommodating a posteriorly situated connecting rod 40 so as to swivel about an axis 14. The axis 14 is spaced apart from the axis of the joint 15, and therefore, during a rotation of the support part 10 about the axis of the joint 15, the connecting rod 40 executes a path movement via its upper end along the path of the axis 14.

FIG. 2 shows the prosthesis in a side view without the rear connecting rod 40; an upper axis 31 for the upper bearing point of the damping device 30 and a lower axis 51 for a lever, which is described in the following, are apparent on the distal prosthesis component 20. Both axes 31, 51 are displaceably mounted in or on the distal prosthesis component 20, and therefore both the separation as well as the position of the particular axes 31, 51 can be adjusted and can be fixed in the set position.

FIG. 3 shows a sectional view of the prosthesis according to FIG. 1. The shell-like support part 10 can be designed for receiving an upper-arm stump, a below knee stump, or an above knee stump. The connecting rod receptacle 13 is apparent on the rear, posterior wall portion of the support part 10, which is recognized for receiving a bolt for forming a bearing point 41 for hingedly fastening the rear connecting rod 40 to the support part 10. The connecting rod 40 is fixed in its proximal region 41, more precisely at its proximal end, to the bearing point 140 of the support part 10. At the opposite end, the connecting rod 40 is mounted on a lever 50 in a distal region 42, i.e., via its distal end in the exemplary embodiment shown. The lever 50 is mounted on the distal prosthesis element 20 about a swivel axis 51 and is designed as a double-armed lever. The rear connecting rod 40 forms a bearing point 160 with the lever 50, and therefore the lever 50 can swivel about the axis 16 during a displacement of the connecting rod 40.

The damping unit 30 is fixed at the lower bearing point 320 on the lever end that faces away from the connecting rod 40 and lies on the other side of the axis 51. The lever 50 itself is swivel-mounted at the bearing point 510 within the hollow space 25 formed by the distal prosthesis element 20. The bearing point 320 on the lever 50 allows for a swinging motion between a piston rod 35 and the lever 50, and therefore the piston rod can execute a linear movement during a rotation about the axis 51, and therefore the piston 34—which is situated within a cylinder 33—of the damping unit 40 designed as a hydraulic damper can execute a displacement movement. The upper, proximal end of the damping device 30 is mounted at the upper bearing point 310 so as to swivel about the axis 31.

In addition to the fixed correlation of the components to one another, as shown in FIG. 3, it is possible that the bearing points 140, 160, 320 are embodied so as to be displaceable, and therefore the particular components can be fixed in a swiveling manner at different positions. In particular, the interspacings of the bearing points 160, 320 of the connecting rod 40 or the damping unit 30 relative to the swivel axis 51 situated between the two bearing points 160, 320 can be changed, and therefore the transmission ratio changes. It is also possible to arrange connecting rods 40 having different lengths between the bearing points 140, 160 or to design the connecting rod 40 to be variable in length, for example, via threaded sleeves, components which are displaceable relative to one another, a telescopic embodiment, or via intermediate pieces. As a result, it is possible to implement the characteristics of the damping, a displacement of a reversal point of the damping device 30, and other force transmissions.

FIG. 4 shows the embodiment of the prosthesis in an angled position, in which the support part 10 is flexed by approximately 90° with respect to a maximally extended position relative to the distal prosthesis element 20. Due to the path curve of the upper bearing point 140 of the connecting rod 40, the connecting rod 40 is swiveled about the axis 14, on the one hand, and, on the other hand, the distal end moves downward, which causes the lever 50 to swivel about the swivel axis 51 and the opposite lever end to be displaced upwardly in the direction of the upper bearing point 310. As a result, the piston rod 35 is retracted into the hydraulic damper and the piston 34 is displaced within the cylinder 33.

In FIG. 5, the prosthesis is shown in a maximally flexed position. The support part 10 is maximally swiveled about the axis of the joint 15, and therefore the indicated receiving element 60 in the form of a femoral shaft rests against the back side of the distal prosthesis element 20. Due to the reversal of motion of the upper bearing point 140, the connecting rod 40 is displaced back in the proximal direction, and therefore the lever 50 was swiveled in the opposite direction, i.e., in the clockwise direction, proceeding from the position according to FIG. 4. A reversal of motion of the piston 34 in the damping device 30 therefore results. An adjusted application of resistance can be provided by selecting a different resistance of overflow valves in the particular direction of motion. In a movement up to the position according to FIG. 4, i.e., during a flexion from an extended position into an approximately 90° position, the piston 34 travels upward. In the position according to FIG. 4, the piston has reached its maximum position; in the reversal of motion upon a further flexion or an extension, the piston 34 is moved downward and can provide, for example, a lesser resistance to a further flexion than during a motion in the upward direction.

Instead of a damping device 30, it is possible to provide a spring device, an actuator device, for example, in the form of a motor, or a combination of at least two of these devices, within the hollow space 25. The lever 50 can be variable in length, and the piston rod 35 can also be variable in length, for example, by means of threaded sleeves. The connecting rod 40 can also be designed to be variable in length.

In addition to a displacement of the bearing points 140, 160, 320, it is also possible to embody the bearing points 520, 310 of the lever 50 and the damping device 30 to be displaceable, i.e., to situate the bearing points 510, 310 on the distal prosthesis component 20 at different points and fix said bearing points there in order to influence the dynamic and static configuration of the prosthesis.

In the embodiment according to FIGS. 1 to 5, the prosthesis is designed as an AKP (above knee prosthesis), in particular, the embodiment is suitable for patients having a knee exarticulation, since the stump can extend into the support part 40, and therefore the axis of the joint 15 of the swivel joint 70, which is formed between the support part 10 and the distal prosthesis component 20, can be situated in the region of the natural axis of the joint of the healthy leg. The axis of the joint 15 can be adjusted individually on the support part 10 or directly on the receiving element 50 by means of the positioning of the bearing points 71, 72. The shell-shaped lower part in the form of the distal prosthesis component 20 partially surrounds the support part 10 and forms corresponding bearing points on the medial and lateral side walls. Since knee exarticulation prostheses cannot be adjusted in the proximal region using pyramidal receiving elements, in order to adjust the dynamic and static configuration of the prosthesis, it is necessary that the bearing points be displaceable in order to adjust a forward displacement and a rearward displacement and, therefore, the prosthesis configuration. It is possible, for example, to design the lever 50 itself to comprise multiple components and to be able to swivel, and therefore, instead of the straight, double-armed embodiment as shown in FIG. 3, an angled arrangement of the two lever legs, which are positioned on either side of the swivel axis 51, sets in. Due to an angular position of the particular lever legs, it is possible, on the one hand, to change the distance between the bearing points 160, 320 of the connecting rod 40 and the piston rod 35 and, in addition, to allow for an effective change in the reversal points and, therefore, an effective variation of the damping properties.

FIG. 6 shows the dependence of the damper force curve of the joint angle, in particular the knee angle, at different lengths of the hydraulic damper. In the upper depiction, the upper curve shows a damping progression for a long connecting rod 40, the middle curve shows a shorter connecting rod 40, and the bottom curve shows the shortest connecting rod 40. Depending on the length of the connecting rod, the reversal point of the piston is reached at different angular positions. The longer the connecting rod 40 is, the earlier the reversal point, i.e., the lowest point of the curve, is reached. The damping decreases in the region of the reversal point, and therefore it is advantageous if the dead center of the piston is reached in the region of a flexion of 90°.

The lower depiction shows the damping curve for differently situated axes and interspacings as well as leverage ratios. It is apparent that different damping properties are present at different knee angles.

The extension stop of the prosthesis can be adjusted by changing the lever lengths, in particular the connecting rod length, and the position of the piston rod 35. This can be achieved at the damping unit 30, for example, at the hydraulic damper, by adjusting the length of the piston rod 35 or by adjusting the length of the connecting rod 40. It is also possible to achieve a corresponding variation of the extension stop and of the maximum flexion angle and/or extension angle by means of a correlation of the lever legs of the lever 50, which are mounted so as to be rotatable relative to one another.

The damping properties of the damping device 30 are preferably set via the knee angle in such a way that the damping properties are optimized for the functionality of the prosthesis. In the region of the dead center or the reversal point of the piston, the damping can be increased, and therefore the damping moments or damping torques in the swivel joint 70 do not abruptly drop off.

FIG. 7 shows a schematic depiction of the components according to the arrangement of the FIGS. 3 to 5. It is apparent that the axis of the joint 15 is situated proximal to the distal end of the support part 10, and therefore a simple length correction, in the case of long stumps, can take place, since the bearing points can be situated virtually anywhere on the support part 10 and the position of the axis of the joint 15 can be adapted to that of the natural axis of the joint. In addition to the embodiment having a knee exarticulation prosthesis, which is shown, it is also possible to position such prostheses on lower legs or upper arms. The lever 50 is designed as a double-armed lever, the rear connecting rod 40 is situated at one lever end, and the damping device 30, likewise together with a spring device or an actuator unit, is situated at the opposite lever end of the double-armed lever 50 which is displaceable about the swivel axis 51. The bearing points of both the lever 50 and the damping unit 30 and/or of the connecting rod 40 can be displaceable.

FIG. 8 shows one variant of the arrangement comprising a single-armed lever 50, in which the bearing points 160, 320 of the connecting rod 40 lie on the same side of the swivel axis 51. It is possible to change the displacement and force transmission ratios by way of an interspacing of the bearing points 160, 320 with respect to one another and by way of the adjustment of the interspacing with respect to one another. In the exemplary embodiment shown, the upper bearing point 310 of the damping device 30 lies above the swivel axis 51 and the lever 50.

One variant of the invention is shown in FIG. 9, which a similar configuration as FIG. 8, although the second bearing point 310 of the damping unit 30 is not situated above the lever 50, but rather below the lever on the distal prosthesis component 20. Such an arrangement is meaningful when the swivel axis 51 is situated proximally relatively far in the distal prosthesis element 30 and there is only a small distance to the support part 10. The bearing points 510, 320, 310 and 160 can be designed to be displaceable or changeable in this case as well.

One variant of the embodiment according to FIG. 9 is depicted in FIG. 10, in which the distal bearing point 310 is situated further posteriorly, the bearing point 510 of the lever is situated very far upward and, due to the relatively small distances between the bearing point 320 on the lever and the bearing point 160 of the connecting rod 40, a low force transfer and displacement results.

FIG. 11 shows one further variant having an intersecting extension of the longitudinal axes of the connecting rod 40 and the damping device 30. In this case as well, the lever 50 is designed as a single-armed lever, and the bearing point 510 lies in the posterior region at the distal end of the distal prosthesis component 20. If the swivel joint is flexed, the lever 50 moves downward in the counterclockwise direction, whereby the damping device 30 is tensile loaded; during an extension motion, a compressive force acts on the damping device 30.

One further variant comprising a single-armed lever is depicted in FIG. 12; the bearing point 510 of the lever 50 lies posteriorly to the axis of the joint 15, and the free lever end extends in the anterior direction and swivels in the counterclockwise direction when a flexion is executed. The variant according to FIG. 12 allows for an increase in displacement for the damping device 30. The double arrows indicate the possible displaceability along the longitudinal extension of the lever 50.

One further variant is depicted in FIG. 13, in which the damping device 30 is situated not on the lever 50, but rather at the distal region 42 of the connecting rod. The lever 50 effectuates a guidance of the connecting rod 40 and the damping device 30; the bearing point 320 lies on the connecting rod 40 and can be displaceably fixed there. A force transmission or displacement can take place via the angular orientation of the longitudinal extension of the damper device 30.

FIG. 14 shows one variant of FIG. 13, in which the damping device 30 is likewise fastened to the connecting rod 40, although in a variant in which the bearing point 310 is positioned on the distal prosthesis component 20 underneath, i.e., distally with respect to the bearing point 320 of the piston rod 35 on the rear connecting rod 40. During a flexion, the damping device 30 is compression loaded and, during an extension, said damping device is conversely tensile loaded.

In FIGS. 1 to 5, in particular, it is clear that the connecting rod 40 at least partially closes the posterior side of the hollow space 25 which is formed by the tubular distal prosthesis element 20. A cutout 26 is provided in the distal prosthesis element 20, in which the material of the wall has been removed, and therefore a curved contour—in the side view—results, as shown in FIG. 2. As a result, it is possible that the support part 10, together with the receiving element 60 fastened thereto or formed thereon, can execute a flexion which goes beyond 90°. This is depicted in FIG. 5. The connecting rod 40 is situated posteriorly in front of the cutout 26 and forms a cover for the damping unit 30 and the mechanics and can be used as a contour-supplementing component of the prosthesis.

The damping unit 30, optionally in combination with a spring unit or an actuator unit, which is designed as a motor and can provide a supporting or braking effect for the prosthesis joint, can be controlled purely mechanically. Alternatively, it is possible to carry out a control via a knee angle sensor and a torque sensor at the ankle in one embodiment as a knee exarticulation prosthesis. Likewise, it is possible that the control is carried out via an inertial sensor in the distal prosthesis element 20 in combination with a hydraulic system which blocks under load. In addition to a combination with a hydraulic system which blocks under load, an inertial sensor can take place in the distal prosthesis element, in combination with a torque measurement at the swivel joint 70, a force measurement at the piston rod or the bearing points of the damping unit 30, or via a pressure measurement in the hydraulic circuit. Likewise, it is possible to provide inertial sensors in the support part, on the receiving element, and in the distal prosthesis element. Gyroscopes and acceleration sensors, and a combination of both sensors, can be used as inertial sensors. Force sensors can be designed as strain gauges, pressure sensors as piezoelements, and angular sensors as Hall sensors.

Due to the embodiment of the prosthesis comprising the connecting rod in combination with the lever, the stroke in the hydraulic unit or in the actuator can be substantially reduced. As a result of a reduction of the stroke at the hydraulic system, the piston rod and the housing can be shortened by the amount of the reduced stroke, and therefore the hydraulic system overall can be shortened by twice the amount of the reduced stroke. This is a very great advantage in confined spatial conditions, in particular.

Due to the change in the length of the connecting rod or due to the extension or shortening of the hydraulic system or the angular position of the lever arms with respect to one another, the position of the support part 10 and, therefore, of a possible prosthesis socket and, therefore, the prosthesis configuration, can be changed.

Due to the embodiment of the support part as a shell, it is possible to define end-loadable stumps using new socket concepts and to accommodate the stump directly in the support part 10. The positioning of the swivel axis 70 on the support part proximally to the distal end of the support part makes it possible to displace the axis of the joint 15 in the proximal direction on the stump or at least as close as possible to the stump. As a result, the moment of inertia is improved due to the proximal fulcrum or the proximal axis of the joint 15, and the patient is able to subjectively more easily sense the prosthesis.

FIG. 15 shows one variant comprising a double-armed lever 50, in which a first lever part 52 is connected to a second lever part 53 so as to swivel about the swivel axis 51. Radial arms 56, 57 are situated on each lever part 52 or 51, which have threads or through-holes and between which, for example, screws 54, 55 are situated. The screws 54 or 55 are setting screws; one of the screws pulls the radial arms 56, 57 toward one another, while the other screw presses in the opposite direction, and therefore a preloading of the setting can take place. In the position shown in FIG. 15, the particular bearing points 160, 320 are maximally separated from one another and the lever arms 52, 53 are aligned with each other.

FIG. 16 shows the lever 50 in a swiveled position; the clamping screw 54 has been rotated in such a way that the angles 56, 57 are displaced toward each other, which effectuates a displacement and swiveling of the two lever parts 52, 53 with respect to one another. The distance between the bearing points 160, 320 has been reduced, and the position of the non-illustrated connecting rod has been varied with respect to the damper, which is also not shown.

FIG. 17 shows one variant of the setting in which, instead of a screw arrangement, retaining holes 58, which are situated around the swivel axis 51 in the shape of a circle, are situated in both lever parts 52, 53. An interlocked locking in the desired angular position can be achieved via an interlocking element, for example, a bolt or the like, depending on the orientation of the particular retaining holes 58 with respect to one another.

1. A prosthesis, comprising:
a support part;
a swivel joint is fastened or formed on the support part;
a distal prosthesis component is fastened on the support part via the swivel joint, the swivel joint allowing for a flexion and an extension of the distal prosthesis component about an axis of the joint relative to a receiving element or the support part;
at least one of a damping device, a spring device and an actuator device positioned between the distal prosthesis component and the support part, the at least one of the damping device, spring device and actuator device being mounted on the distal prosthesis component with a first bearing point and mounted on a lever or a connecting rod with a second bearing point, the lever being mounted on the distal prosthesis component so as to swivel about a swivel axis, and the connecting rod being mounted on the lever via a distal portion and mounted on the support part via a proximal portion.

2. The prosthesis as claimed in claim 1, wherein the receiving element for receiving a stump is formed on or fastened to the support part.

3. The prosthesis as claimed in claim 1, wherein the support part is configured to receive a stump.

4. The prosthesis as claimed in claim 1, wherein the lever comprises a double-armed lever and the swivel axis is positioned between the at least one of the damping device, spring device and/or actuator device and the connecting rod.

5. The prosthesis as claimed in claim 1, wherein the lever comprises a single-armed lever, and the connecting rod and the at least one of the damping device, spring device and actuator device is mounted on the lever at a distance from the swivel axis.

6. The prosthesis as claimed in claim 1, wherein at least one of the connecting rod and the at least one of the damping device, spring device and actuator device are displaceably mounted on the lever.

7. The prosthesis as claimed in claim 1, wherein at least one of the at least one of the damping device, spring device and/or actuator device, the connecting rod and the lever are designed to be variable in length.

8. The prosthesis as claimed in claim 1, wherein the distal prosthesis component forms a hollow space in which the lever and the at least one of the damping device, spring device and actuator device are situated.

9. The prosthesis as claimed in claim 8, wherein the connecting rod at least partially covers an access to the hollow space.

10. The prosthesis as claimed in claim 8, wherein the connecting rod supplements a contour of the distal prosthesis component.

11. The prosthesis as claimed in claim 1, wherein the axis of the joint extends through the support part or the receiving element.

12. The prosthesis as claimed in claim 1, wherein the distal prosthesis component is mounted directly on the support part or receiving element at at least one bearing point, and the at least one bearing point is positioned medially or laterally on the support part or the receiving element.

13. The prosthesis as claimed in claim 1, wherein the axis of the joint is positioned in a region of a natural axis of the joint of a limb.

14. The prosthesis as claimed in claim 1, wherein the connecting rod is displaceably mounted on the support part or the receiving element.

15. The prosthesis as claimed in claim 1, wherein the axis of the joint is positioned proximal of a distal end of the support part or the receiving element.

16. The prosthesis as claimed in claim 1, wherein the first and second bearing points displaceably formed on at least one of the distal prosthesis component and the lever.

17. The prosthesis as claimed in claim 1, further comprising a blocking device to block at least one of the flexion and the extension of the distal prosthesis component.

18. A prosthesis, comprising:
a support part;
a swivel joint positioned on the support part;
a distal prosthesis component connected to the support part via the swivel joint, the swivel joint providing flexion and extension movement of the distal prosthesis component about a rotation axis of the swivel joint;
a lever connected to the distal prosthesis component and movable about a swivel axis;
a connecting rod having distal and proximal portions, the distal portion being connected to the lever and the proximal portion being connected to the support part;
at least one of a damping device, a spring device and an actuator device positioned between the distal prosthesis component and the support part, the at least one of the damping device, spring device and actuator device being connected to the distal prosthesis component at a first bearing point and connected to the lever or connecting rod at a second bearing point.

19. The prosthesis as claimed in claim 18, further comprising a receiving element to receive a stump, the receiving element being formed on or fastened to the support part.

20. The prosthesis as claimed in claim 18, wherein the support part is configured to receive a stump.
